(19) United States
(12) Patent Application Publication
(10) Pub. No.: US 2008/0027146 A1
(43) Pub. Date: Jan. 31, 2008

(54) METHODS AND COMPOSITIONS FOR DETECTING AND MODULATING PERIODONTAL DISORDERS AND PERIODONTAL DISEASES

(75) Inventors: Joseph P. Fiorellini, Merion Station, PA (US); Myron Nevins, Swampscott, MA (US); David M. Kim, Cambridge, MA (US); Marco F. Ramoni, Boston, MA (US)

Correspondence Address: BANNER & WITCOFF, LTD., 28 STATE STREET, 28th FLOOR, BOSTON, MA, 02109-9601, US

(73) Assignee: President and Fellows of Harvard College, Cambridge, MA (US)

(21) Appl. No.: 11/774,095

(22) Filed: Jul. 6, 2007

Related U.S. Application Data
(63) Continuation of application No. PCT/US06/00755, filed on Jan. 9, 2006.
(60) Provisional application No. 60/645,705, filed on Jan. 21, 2005.
(60) Provisional application No. 60/642,244, filed on Jan. 7, 2005.

(51) Int. Cl. C12Q 1/37; A61K 45/00; A61P 43/00; G01N 33/50; C12Q 1/02

(52) U.S. Cl. 514/789; 435/23; 435/29; 436/86; 436/87

(57) ABSTRACT

The invention provides genetic markers of periodontal disorders and/or periodontal diseases. The present invention provides methods and compositions to aid in the detection, diagnosis, treatment and monitoring of periodontal disorders and/or periodontal diseases.

### RELATED U.S. APPLICATIONS

The present application claims priority from International Application No. PCT/US2006/000755, filed Jan. 9, 2006, which designates the United States; which claims priority from U.S. Provisional Application No. 60/645,705, filed Jan. 21, 2005; and from U.S. Provisional Application No. 60/642,244, filed Jan. 7, 2005; each of which is hereby incorporated by reference in its entirety for all purposes.

### FIELD OF THE INVENTION

Embodiments of the present invention relate to the detection, diagnosis, treatment and monitoring of periodontal disorders and/or periodontal diseases.

### BACKGROUND

Periodontal disease refers to the inflammatory response of gingival and surrounding connective tissue which may result from bacterial or plaque accumulations on teeth (see Section 5, Introduction to Infectious Disease, Baron, S. Medical Microbiology, 4th ed., University of Texas Medical Branch, Galveston, Tex., 1996). Periodontal disease is divided into two main groupings: gingivitis, which is typically described as inflammation and/or bleeding of the gingival or gum tissues in the absence of bone loss; and periodontitis, which is typically described as the pathologic loss of tissue between the tooth and the gingival tissue. Id. Periodontitis occurs when a plaque-induced inflammatory response in the tissue results in loss of collagen attachment of one or more teeth to the bone. Id.

Bacteria are essential for initiation and progression of periodontitis, but microbial factors alone do not predict the presence or the extent of periodontitis (Hart (1997)). Moreover, predicting the course of destruction can be a difficult task due to the variable nature of the disease process and the significant influence of environmental factors. Existing diagnostic procedures and guides for treatment have little benefit in predicting individual patient's future periodontal status (Newman (1997)).

Currently, treatment of periodontal diseases and disorders is mechanical and surgical in nature and frequently includes scaling and root planing to remove calculus deposits. Such mechanical treatments do not affect the underlying cause of the disease or disorder, however. Although antibiotics have been used as an adjunct therapy, results have been disappointing because the antibiotic may not completely eliminate bacteria responsible for inflammation, leading to a recurrence of the periodontal disease or disorder.

Periodontitis is a serious health problem in the United States today. According to data from the third National Health and Nutrition Examination Survey (NHANES III), periodontitis is so prevalent in the U.S. adult population that at least 35% of U.S. adults aged 30 to 90 have periodontitis, with approximately 21.8% having an early form and approximately 12.6% having a moderate to advanced form (Albander (1999)). In 1999, dentists in private practice performed a total of 28.5 million periodontal procedures and the total expenditure on periodontal and preventive procedures was $14.3 billion (American Dental Association (1999)). The inability of clinicians to determine the future course of a patient's periodontal health based on biological factors leads to inconsistent and somewhat ineffective therapeutic approaches that result in under-and/or over-treatment. Many clinicians find that current routine diagnostic methods for detecting periodontitis are nothing more than recordings of the history of previous destructive disease. This results in patients being under-treated based on the lack of objective findings at the time of examination, or over-treated in an attempt to achieve the best possible level of periodontal health. Accordingly, identification of risk factors, such as genetic markers, associated with periodontal diseases and periodontal disorders are needed.

### SUMMARY OF THE INVENTION

It is therefore an object of this invention to provide genetic markers to aid in the detection, diagnosis, treatment and monitoring of periodontal disorders and/or periodontal diseases.

Embodiments of the present invention are based in part on the discovery of genes associated with periodontitis. According to one embodiment of the present invention, one or more of the genes described herein are used as markers for the detection of periodontal diseases and/or periodontal disorders, such as, for example, gingivitis and/or periodontitis. In certain aspects of the invention, one or more of the genes described herein are used as markers for the diagnosis and prognosis of periodontal diseases and/or disorders.

Embodiments of the present invention are directed to methods of treating periodontal diseases or disorders by up-regulating or down-regulating one or more of the markers described herein. These markers may also advantageously be used to identify and/or design novel therapeutic compounds for the treatment of periodontal diseases or disorders. The markers described herein may further be used as markers to predict the efficacy of therapeutic compounds in treating such diseases and disorders in specific individuals.

Embodiments of the present invention are directed to a method for diagnosing a periodontal disorder in an organism, such as a human. According to the method, a biological sample is obtained from the organism. The level of a biomarker in the biological sample is detected. A periodontal disorder is diagnosed based on the presence and/or level of the biomarker detected. According to a certain embodiment of the invention, the biomarker is a member selected from the group consisting of lactotransferrin, V-FOS FBJ murine osteosarcoma viral oncogene homolog B, matrix metalloproteinase 1, matrix metalloproteinase 3, caspase 10, interleukin 24, interferon-induced protein IFI-15k, Rho GTPASE activating protein 8, hypothetical protein MGC5566, desmocollin 1, keratin 2A and transmembrane 7 superfamily member 3.

Embodiments of the present invention are also directed to a method for diagnosing a periodontal disorder in an organism where a biological sample is obtained from the organism. A level of biomarker in the biological sample is detected and compared to the level of biomarker in a chart or database correlating a level of biomarker with a periodontal disorder. The periodontal disorder is than diagnosed when the level of the biomarker in the biological sample corresponds to the level of biomarker that the chart correlates with a periodontal disorder. According to a certain embodiment of the invention, the biomarker is a member selected from the group consisting of lactotransferrin, V-FOS FBJ murine osteosarcoma viral oncogene homolog B, matrix metalloproteinase 1, matrix metalloproteinase 3, caspase 10, interleukin 24, interferon-induced protein IFI-15k, Rho GTPASE activating protein 8, hypothetical protein MGC5566, desmocollin 1, keratin 2A and transmembrane 7 superfamily member 3.

Embodiments of the present invention are also directed to a method for diagnosing a periodontal disorder in an organism where a biological sample is obtained from the organism. A level of biomarker in the biological sample is detected and compared with the level of biomarker in a control sample. The organism is diagnosed with a periodontal disorder when an altered level of the biomarker is detected in the biological sample relative to the control sample. According to a certain embodiment of the invention, the biomarker is a member selected from the group consisting of lactotransferrin, V-FOS FBJ murine osteosarcoma viral oncogene homolog B, matrix metalloproteinase 1, matrix metalloproteinase 3, caspase 10, interleukin 24, interferon-induced protein IFI-15k, Rho GTPASE activating protein 8, hypothetical protein MGC5566, desmocollin 1, keratin 2A and transmembrane 7 superfamily member 3.

According to certain embodiments of the present invention, the level of biomarker can be either increased or decreased in the biological sample relative to the control sample. A further embodiment of the present invention is directed to a panel of biomarkers for detecting a periodontal disorder in an organism comprising two or more biomarkers selected from the group consisting of lactotransferrin; V-FOS FBJ murine osteosarcoma viral oncogene homolog B, matrix metalloproteinase 1, matrix metalloproteinase 3, caspase 10, interleukin 24, interferon-induced protein IFI-15k, Rho GTPASE activating protein 8, hypothetical protein MGC5566, desmocollin 1, keratin 2A and transmembrane 7 superfamily member 3.

Embodiments of the present invention are still further directed to a method for monitoring a periodontal disorder in an organism where a first biological sample is obtained from the organism at a first point in time. A second biological sample is obtained from the organism at a second point in time. A level of a biomarker is determined in the first and second biological samples. The level of biomarker in the first and second biological samples is compared. A decrease of the level of the biomarker in the second sample relative to the first sample indicates a decrease in a periodontal disorder in the organism. Biomarkers are selected from the group consisting of lactotransferrin; V-FOS FBJ murine osteosarcoma viral oncogene homolog B, matrix metalloproteinase 1, matrix metalloproteinase 3, caspase 10, interleukin 24, interferon-induced protein IFI-15k, Rho GTPASE activating protein 8, and hypothetical protein MGC5566. According to an aspect of the present invention, an increase of the level of the biomarker in the second sample relative to the first sample indicates a decrease in a periodontal disorder in the organism. Biomarkers are selected from the group consisting of desmocollin 1, keratin 2A and transmembrane 7 superfamily member 3.

Embodiments of the present invention are still further directed to a method of treating a periodontal disorder in an organism. According to the method, a cell is contacted with an agent that down-regulates a biomarker selected from the group consisting of lactotransferrin, V-FOS FBJ murine osteosarcoma viral oncogene homolog B, matrix metalloproteinase 1, matrix metalloproteinase 3, caspase 10, interleukin 24, interferon-induced protein IFI-15k, Rho GTPASE activating protein 8, and hypothetical protein MGC5566, in a manner to reduce symptoms associated with the periodontal disorder. According to an alternate treatment method, a cell is contacted with an agent that up-regulates a biomarker selected from the group consisting of desmocollin 1, keratin 2A and transmembrane 7 superfamily member 3, in a manner to reduce symptoms associated with the periodontal disorder.

Additional further embodiments of the present invention are directed to a method for screening compounds useful in treating a periodontal disorder in an organism wherein a cell is contacted with a test compound followed by determining whether the test compound up-regulates a biomarker selected from the group consisting of desmocollin 1, keratin 2A and transmembrane 7 superfamily member 3. Alternate screening embodiments are directed to determining whether the test compound down-regulates a biomarker selected from the group consisting of lactotransferrin, V-FOS FBJ murine osteosarcoma viral oncogene homolog B, matrix metalloproteinase 1, matrix metalloproteinase 3, caspase 10, interleukin 24, interferon-induced protein IFI-15k, Rho GTPASE activating protein 8, and hypothetical protein MGC5566.

It will be recognized by the person of ordinary skill in the art that the markers, compounds, compositions and methods disclosed herein provide significant advantages over prior technology. Compounds, compositions and methods can be designed or selected to relieve and/or alleviate symptoms in a patient suffering from one or more periodontal diseases and/or periodontal disorders. These and other aspects and examples are described below. Other features and advantages of the invention will be apparent from the following detailed description and claims.

### DETAILED DESCRIPTION OF THE INVENTION

The present invention provides genetic markers associated with periodontal diseases and/or periodontal disorders. Embodiments of the present invention are based on the discovery that certain genes are differentially expressed (i.e., up-regulated or down-regulated) in individuals suffering from a periodontal disorder. These genes can be used as markers of one or more periodontal diseases and/or periodontal disorders. In certain embodiments, the present invention is directed to detection, diagnosis, treatment and/or monitoring periodontal diseases and/or periodontal disorders.

As used herein, the term “marker” is intended to include, but is not limited to, one or more DNA sequences (e.g., genomic DNA), RNA sequences (e.g., mRNA) or peptides (e.g., polypeptides and/or proteins) corresponding to one or more of the genes described herein that is differentially expressed in a periodontal disease and/or a periodontal disorder. Such markers are described, for example, in FIGS. 1-8, Table 1 and Examples II and III. In one embodiment, the presence or absence of one or more markers of the invention is used to diagnose one or more periodontal diseases and/or periodontal disorders in an individual.

As used herein, the terms “periodontal disease” and “periodontal disorder” refer to inflammatory responses and/or degeneration of gingival and surrounding connective tissue and bone. Periodontal diseases and disorders include, but are not limited to, gingivitis and periodontitis. Periodontal diseases and disorders have also been associated with cardiovascular disease (Beck et al. (1999) Am. Heart J. 13:S528; Beck et al. (2001) Arterioscler. Thromb. Vasc. Biol. 21:1816).

Gingivitis is clinically manifested by one or more of: inflammation of gingival and/or gum tissues; bleeding of gingival and/or gum tissues; and the presence of pseudopockets. Gingivitis is divided into subdivisions including, but not limited to: plaque-associated gingivitis; chronic gingivitis; acute necrotizing ulcerative gingivitis; gingivitis associated with systemic conditions or medications (e.g., hormone-induced gingival inflammation, drug-influenced gingivitis, linear gingival erythema, and the like); and gingival manifestations of systemic diseases and monocutaneous lesions (e.g., bacterial gingivitis, viral gingivitis, fungal gingivitis, blood dyscrasias (such as acute monocytic leukemia), and monocutaneous diseases (such as lichen planus, cicatricial pemphigoid) and the like.

Periodontitis may be characterized as early periodontitis, moderate periodontitis or advanced periodontitis. Early periodontitis is clinically manifested by one or more of: bleeding upon probing; the presence of pockets (3 to 4 mm); localized areas of recession; attachment loss (3 to 4 mm); bone loss (e.g., horizontal); and class I furcation invasion areas. Moderate periodontitis is clinically manifested by one or more of: the presence of pockets (4 to 6 mm); the presence of attachment loss (4 to 6 mm); bleeding upon probing; grade I and/or grade II furcation invasion areas; class I tooth mobility; bone loss (e.g., horizontal and/or vertical); and loss of ⅓ of supporting alveolar bone (i.e., crown to root ratio of 1:1). Advanced periodontitis is clinically manifested by one or more of: bleeding upon probing; the presence of pockets (over 6 mm); attachment loss (over 6 mm); grade II and/or grade III furcation invasion areas; class II and/or class III tooth mobility; bone loss (e.g., horizontal and/or vertical); and loss of over ⅓ of supporting alveolar bone (i.e., crown to root ratio of 2:1 or more). Periodontitis is divided into subdivisions including, but not limited to: adult periodontitis (e.g., plaque-associated); early-onset periodontitis (e.g., prepubertal, juvenile, rapidly progressive and the like); periodontitis associated with systemic diseases; necrotizing ulcerative periodontitis; refractory periodontitis; peri-implantitis and the like.

In at least certain examples, compounds described herein can be used in the treatment of periodontal diseases and/or periodontal disorders associated with modulation, e.g., up-regulation and/or down-regulation, of one or more markers of the invention. The language “treatment of periodontal diseases” and “treatment of periodontal disorders” is intended to include the prevention of periodontal diseases and/or periodontal disorders in a subject or an inhibition of the progression of one or more pre-existing periodontal diseases and/or periodontal disorders in a subject. As used herein, the terms “inhibit” and “inhibition” refer to a partial inhibition or a complete inhibition of a periodontal disease and/or a periodontal disorder compared to the condition without treatment such that therapeutic treatment and/or prophylaxis results.

Examples of the types of periodontal diseases and/or periodontal disorders intended to be encompassed by the present invention include, but are not limited to, those associated with inflammation of oral tissues such as gingivitis and periodontitis. Periodontal diseases and/or periodontal disorders can further include cardiovascular disorders such as atherosclerosis and restinosis.

Diagnostic Assays

An exemplary method for detecting the presence or absence of a periodontal disease and/or periodontal disorder in a biological sample involves obtaining a biological sample from a test subject and contacting the biological sample with a compound or an agent capable of detecting one or more of the markers of periodontal diseases and/or periodontal disorders described herein, e.g., marker nucleic acid (e.g., mRNA, genomic DNA) or marker peptide (e.g., polypeptide or protein) encoded by the marker nucleic acid such that the presence of a marker nucleic acid or marker peptide encoded by the nucleic acid is detected in the biological sample. In one embodiment, an agent for detecting marker mRNA or genomic DNA is a labeled nucleic acid probe capable of hybridizing to marker mRNA or genomic DNA. The nucleic acid probe can be, for example, a full-length marker nucleic acid or a portion thereof. Other suitable probes for use in the diagnostic assays of the invention are described herein.

Another agent for detecting marker peptide is an antibody capable of binding to a marker peptide, such as an antibody with a detectable label. Antibodies can be polyclonal or monoclonal. An intact antibody, or a fragment thereof (e.g., Fab or F(ab′)₂) can be used. The term “labeled,” with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

As used herein, the term “biological sample” is intended to include tissues, cells and biological fluids isolated from a subject, as well as tissues, cells and fluids present within a subject. That is, the detection method of the invention can be used to detect marker mRNA, peptide (e.g., protein), or genomic DNA in a biological sample in vitro as well as in vivo. For example, in vitro techniques for detection of marker mRNA include Northern hybridizations and in situ hybridizations. In vitro techniques for detection of marker peptide include enzyme linked immunosorbent assays (ELISAs), Western blots, immunoprecipitations and immunofluorescence. In vitro techniques for detection of marker genomic DNA include Southern hybridizations. In vivo techniques for detection of marker peptide include introducing into a subject a labeled anti-marker antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. In one embodiment biological sample is a serum sample, saliva sample or a biopsy sample (e.g., a gingival biopsy sample) isolated by conventional means from a subject.

In another embodiment, the methods further involve obtaining a control biological sample from a control subject, contacting the control sample with a compound or agent capable of detecting marker peptides, mRNA, or genomic DNA, such that the presence of marker peptide, mRNA or genomic DNA is detected in the biological sample, and comparing the presence of marker peptides, mRNA or genomic DNA in the control sample with the presence of marker peptide, mRNA or genomic DNA in the test sample. Alternatively, the presence of marker peptide, mRNA or genomic DNA in the test sample can be compared with information in a database or on a chart to result in detection or diagnosis.

The invention also encompasses kits for detecting the presence of one or more markers associated with a periodontal disease or a periodontal disorder in a biological sample. For example, the kit can comprise a labeled compound or agent capable of detecting marker peptide or mRNA in a biological sample; means for determining the amount of marker in the sample; and means for comparing the amount of marker in the sample with a standard. The compound or agent can be packaged in a suitable container. The kit can further comprise instructions for using the kit to detect marker peptide or nucleic acid.

Prognostic Assays

The diagnostic methods described herein can furthermore be utilized to identify subjects having or at risk of developing a periodontal disease and/or periodontal disorder associated with up-regulation or downregulation of one or more of the markers described herein. For example, the assays described herein, such as the preceding diagnostic assays or the following assays, can be utilized to identify a subject having or at risk of developing a periodontal disease and/or a periodontal disorder, such as gingivitis and/or periodontitis.

The prognostic assays described herein can be used to determine whether a subject can be administered an agent (e.g., an agonist, antagonist, peptidomimetic, protein, peptide, nucleic acid, small molecule, or other drug candidate) to treat a periodontal disease and/or periodontal disorder associated with up-regulation or down-regulation of one or more of the markers described herein. For example, such methods can be used to determine whether a subject can be effectively treated with an agent for treating, ameliorating or reducing one or more symptoms associated with gingivitis and/or periodontitis. Thus, the present invention provides methods for determining whether a subject can be effectively treated with an agent for a periodontal disorder and/or periodontal disease.

The methods of the invention can also be used to detect genetic alterations in a marker gene, thereby determining if a subject with the altered gene is at risk for developing a periodontal disorder and/or periodontal disease characterized by misregulation in a marker protein activity or nucleic acid expression, such as gingivitis and/or periodontitis. In certain embodiments, the methods include detecting, in a sample of cells from the subject, the presence or absence of a genetic alteration characterized by an alteration affecting the integrity of a gene encoding a marker peptide and/or a marker gene. For example, such genetic alterations can be detected by ascertaining the existence of at least one of: 1) a deletion of one or more nucleotides from one or more marker genes; 2) an addition of one or more nucleotides to one or more marker genes; 3) a substitution of one or more nucleotides of one or more marker genes, 4) a chromosomal rearrangement of one or more marker genes; 5) an alteration in the level of a messenger RNA transcript of one or more marker genes; 6) aberrant modification of one or more marker genes, such as of the methylation pattern of the genomic DNA; 7) the presence of a non-wild type splicing pattern of a messenger RNA transcript of one or more marker genes; 8) a non-wild type level of a one or more marker proteins; 9) allelic loss of one or more marker genes; and 10) inappropriate post-translational modification of one or more marker proteins. As described herein, there are a large number of assays known in the art which can be used for detecting alterations in one or more marker genes.

In certain embodiments, detection of the alteration involves the use of a probe/primer in a polymerase chain reaction (PCR) (see, e.g., U.S. Pat. Nos. 4,683,195 and 4,683,202), such as real-time PCR, anchor PCR, recursive PCR or RACE PCR, or, alternatively, in a ligation chain reaction (LCR) (see, e.g., Landegran et al. (1988) Science 241:1077; Prodromou and Pearl (1992) Protein Eng. 5:827; and Nakazawa et al. (1994) Proc. Natl. Acad. Sci. USA 91:360), the latter of which can be particularly useful for detecting point mutations in a marker gene (see Abravaya et al. (1995) Nucleic Acids Res. 23:675). This method can include the steps of collecting a sample of cells from a subject, isolating nucleic acid (e.g., genomic, mRNA or both) from the cells of the sample, contacting the nucleic acid sample with one or more primers which specifically hybridize to a marker gene under conditions such that hybridization and amplification of the marker gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli et al., (1990) Proc. Natl. Acad. Sci. USA 87:1874), transcriptional amplification system (Kwoh et al., (1989) Proc. Natl. Acad. Sci. USA 86:1173), Q-Beta Replicase (Lizardi et al. (1988) Bio-Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in one or more marker genes from a sample cell can be identified by alterations in restriction enzyme cleavage patterns. For example, sample and control DNA is isolated, optionally amplified, digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in one or more of the markers described herein can be identified by hybridizing a sample and control nucleic acids, e.g., DNA or RNA, to high density arrays containing hundreds or thousands of oligonucleotides probes (Cronin et al. (1996) Human Mutation 7: 244; Kozal et al. (1996) Nature Medicine 2:753). For example, genetic mutations in a marker nucleic acid can be identified in two dimensional arrays containing light-generated DNA probes as described in Cronin, M. T. et al. supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence a marker gene and detect mutations by comparing the sequence of the sample marker gene with the corresponding wild-type (control) sequence. Examples of sequencing reactions include those based on techniques developed by Maxam and Gilbert ((1977) Proc. Natl. Acad. Sci. USA 74:560) or Sanger ((1977) Proc. Natl. Acad. Sci. USA 74:5463). It is also contemplated that any of a variety of automated sequencing procedures can be utilized when performing the diagnostic assays ((1995) Biotechniques 19:448), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147).

Other methods for detecting mutations in a marker gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes formed by hybridizing (labeled) RNA or DNA containing the wild-type marker sequence with potentially mutant RNA or DNA obtained from a tissue sample. The double-stranded duplexes are treated with an agent which cleaves single-stranded regions of the duplex such as which will exist due to base pair mismatches between the control and sample strands. For instance, RNA/DNA duplexes can be treated with RNase and DNA/DNA hybrids treated with S1 nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al. (1988) Proc. Natl. Acad. Sci. USA 85:4397; Saleeba et al. (1992) Methods Enzymol. 217:286. In one embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in marker cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657). According to an exemplary embodiment, a probe based on a marker sequence, e.g., a wild-type marker sequence, is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations in marker genes. For example, single strand conformation polymorphism (SSCP) may be used to detect differences in electrophoretic mobility between mutant and wild type nucleic acids (Orita et al. (1989) Proc. Natl. Acad. Sci. USA 86:2766, see also Cotton (1993) Mutat. Res. 285:125; and Hayashi (1992) Genet. Anal. Tech. Appl. 9:73). Single-stranded DNA fragments of sample and control marker nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility (Keen et al. (1991) Trends Genet. 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys. Chem. 265:12753).

Examples of other techniques for detecting point mutations include, but are not limited to, selective oligonucleotide hybridization, selective amplification or selective primer extension. For example, oligonucleotide primers may be prepared in which the known mutation is placed centrally and then hybridized to target DNA under conditions which permit hybridization only if a perfect match is found (Saiki et al. (1986) Nature 324:163; Saiki et al. (1989) Proc. Natl. Acad. Sci. USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nuc. Acids Res. 17:2437) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6: 1). It is anticipated that in certain embodiments amplification may also be performed using Taq ligase for amplification (Barany (1991) Proc. Natl. Acad. Sci. USA 88:189). In such cases, ligation will occur only if there is a perfect match at the 3′ end of the 5′ sequence making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a periodontal disease and/or periodontal disorder, such as gingivitis or periodontitis.

Furthermore, any cell type or tissue in which one or more of the markers described herein is expressed may be utilized in the prognostic assays described herein.

Therapeutic Methods

Another aspect of the invention pertains to methods of modulating one or more markers of a periodontal disease and/or periodontal disorder for therapeutic purposes. Accordingly, in an exemplary embodiment, the modulatory method of the invention involves contacting a cell with an agent that up-regulates or down-regulates one or more of the markers of the invention. An agent that up-regulates or down-regulates one or more markers of the invention can be an agent as described herein, a naturally-occurring target molecule of one or more markers of the invention, an antibody against a marker of the invention, or other small molecule. As such, the present invention provides methods of treating an individual afflicted with one or more periodontal diseases and/or periodontal disorders. Examples of such disorders are described herein. In one embodiment, the method involves administering an agent (e.g., an agent identified by a screening assay described herein), or combination of agents that inhibit one or more markers of periodontal diseases and/or periodontal disorders.

In one embodiment, the present invention involves a method for treatment of a periodontal disease and/or periodontal disorder which includes the step of administering a therapeutically effective amount of an agent which inhibits the periodontal disease and/or periodontal disorder to a subject in need of such treatment. As defined herein, a therapeutically effective amount of agent (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of an inhibitor can include a single treatment or, preferably, can include a series of treatments. It will also be appreciated that the effective dosage of in used for treatment may increase or decrease over the course of a particular treatment. Changes in dosage may result from the results of diagnostic assays as described herein.

Pharmaceutical Compositions

Methods of administering a compound to an individual include providing pharmaceutically acceptable compositions. In one embodiment, pharmaceutically acceptable compositions comprise a therapeutically effective amount of one or more of the compounds described herein, formulated together with one or more pharmaceutically acceptable carriers (additives) and/or diluents. The pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes for application to the tongue; (2) parenteral administration, for example, by subcutaneous, intramuscular or intravenous injection as, for example, a sterile solution or suspension; (3) topical application, for example, as a cream, ointment or spray applied to the skin; or (4) intravaginally or intrarectally, for example, as a pessary, cream or foam. In one embodiment, the therapeutic compound is administered orally. The compounds of the invention can be formulated as pharmaceutical compositions for administration to a subject, e.g., a mammal, including a human.

The compounds of the invention are administered to subjects in a biologically compatible form suitable for pharmaceutical administration in vivo. By “biologically compatible form suitable for administration in vivo” is meant a compound to be administered in which any toxic effects are outweighed by the therapeutic effects of the compound. The term “subject” is intended to include living organisms such as mammals. Examples of subjects include humans, monkeys, cows, horses, sheep, goats, pigs, dogs, cats, rabbits, mice, rats, frogs, toads and transgenic species thereof. Administration of a therapeutically active amount of the therapeutic compositions of the present invention is defined as an amount effective, at dosages and for periods of time necessary to achieve the desired result. For example, a therapeutically active amount of a compound of the invention may vary according to factors such as the disease state, age, sex, and weight of the individual, and the ability of antibody to elicit a desired response in the individual. Dosage regimes may be adjusted to provide the optimum therapeutic response. For example, several divided doses may be administered daily or the dose may be proportionally reduced as indicated by the exigencies of the therapeutic situation.

The active compound may be administered in a convenient manner such as by oral administration, injection (subcutaneous, intravenous, etc.), inhalation, transdermal application, or rectal administration. Depending on the route of administration, the active compound may be coated in a material to protect the compound from the action of enzymes, acids and other natural conditions which may inactivate the compound.

Compositions suitable for oral use include, but are not limited to, solutions, suspensions, dispersions, ointments, creams, pastes, powders (e.g., tooth powders), toothpastes, gels, liquid dentifrices, lozenges, troches, salve, chewing gum, mouth sprays, pastilles, sachets, mouthwashes, aerosols, tablets, capsules, floss, gingival massage creams, gargle tablets, foodstuffs and the like.

A compound of the invention can be administered to a subject in an appropriate carrier or diluent, co-administered with enzyme inhibitors or in an appropriate carrier such as liposomes. The term “pharmaceutically acceptable carrier” as used herein is intended to include diluents such as saline and aqueous buffer solutions. To administer a compound of the invention by other than parenteral administration, it may be necessary to coat the compound with, or co-administer the compound with a material to prevent its inactivation. Liposomes include water-in-oil-in-water emulsions as well as conventional liposomes (Strejan et al. (1984) J. Neuroimmunol. 7:27). The active compound may also be administered parenterally or intraperitoneally. Dispersions can also be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion. In all cases, the composition must be sterile and must be fluid to the extent that easy syringability exists. It must be stable under the conditions of manufacture and storage and must be preserved against the contaminating action of microorganisms such as bacteria and fungi. The pharmaceutically acceptable carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient (e.g., antibody) plus any additional desired ingredient from a previously sterile-filtered solution thereof.

When the active compound is suitably protected, as described above, the composition may be orally administered, for example, with an inert diluent or an assimilable edible carrier. As used herein “pharmaceutically acceptable carrier” includes any and all solvents, dispersion media, coatings, antibacterial and antifungal agents, isotonic and absorption delaying agents, and the like. The use of such media and agents for pharmaceutically active substances is well known in the art. Except insofar as any conventional media or agent is incompatible with the active compound, use thereof in the therapeutic compositions is contemplated. Supplementary active compounds can also be incorporated into the compositions.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the therapeutic treatment of individuals.

Screening Assays

The present invention provides a method (also referred to herein as a “screening assay”) for identifying modulators, i.e., candidate or test compounds or agents (e.g., peptides, cyclic peptides, peptidomimetics, small molecules, small organic molecules, or other drugs) which have a stimulatory or inhibitory effect on one or more of the markers described herein (i.e., markers of periodontal diseases and/or periodontal disorders).

As used herein, the term “small organic molecule” refers to an organic molecule, either naturally occurring or synthetic, that has a molecular weight of more than about 25 daltons and less than about 3000 daltons, preferably less than about 2500 daltons, more preferably less than about 2000 daltons, preferably between about 100 to about 1000 daltons, more preferably between about 200 to about 500 daltons.

In one embodiment, the invention provides assays for screening candidate or test compounds which are substrates of one or more of the markers of periodontal diseases and/or periodontal disorders described herein. In another embodiment, the invention provides assays for screening candidate or test compounds which bind to or modulate the activity of one or more of the markers of periodontal diseases and/or periodontal disorders described herein.

The test compounds of the present invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. USA 90:6909; Erb et al. (1994) Proc. Natl. Acad. Sci. USA 91:11422; Zuckermann et al. (1994) J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412), or on beads (Lam (1991) Nature 354:82), chips (Fodor (1993) Nature 364:555), bacteria (Ladner U.S. Pat. No. 5,223,409), spores (Ladner U.S. Pat. No. 5,223,409), plasmids (Cull et al. (1992) Proc. Natl. Acad. Sci. USA 89:1865) or on phage (Scott and Smith (1990) Science 249:386); (Devlin (1990) Science 249:404); (Cwirla et al. (1990) Proc. Natl. Acad. Sci. USA 87:6378); (Felici (1991) J. Mol Biol. 222:301); (Ladner supra).

Examples of methods for introducing a molecular library of randomized nucleic acids into a population of cells can be found in the art, for example in U.S. Pat. No. 6,365,344, incorporated herein in its entirety by reference. A molecular library of randomized nucleic acids can provide for the direct selection of candidate or test compounds with desired phenotypic effects. The general method can involve, for instance, expressing a molecular library of randomized nucleic acids in a plurality of cells, each of the nucleic acids comprising a different nucleotide sequence, screening for a cell of exhibiting a changed physiology in response to the presence in the cell of a candidate or test compound, and detecting and isolating the cell and/or candidate or test compound.

In one embodiment, the introduced nucleic acids are randomized and expressed in the cells as a library of isolated randomized expression products, which may be nucleic acids, such as mRNA, antisense RNA, siRNA, ribozyme components, etc., or peptides (e.g., cyclic peptides). The library should provide a sufficiently structurally diverse population of randomized expression products to effect a probabilistically sufficient range of cellular responses to provide one or more cells exhibiting a desired response. Generally at least 10⁶, at least 10⁷, at least 10⁸, or at least 10⁹ different expression products are simultaneously analyzed in the subject methods. In one aspect methods maximize library size and diversity.

The introduced nucleic acids and resultant expression products are randomized, meaning that each nucleic acid and peptide consists of essentially random nucleotides and amino acids, respectively. The library may be fully random or biased, e.g., in nucleotide/residue frequency generally or per position. In other embodiments, the nucleotides or residues are randomized within a defined class, e.g., of hydrophobic amino acids, of purines, etc.

Functional and structural isolation of the randomized expression products may be accomplished by providing free (not covalently coupled) expression product, though in some situations, the expression product may be coupled to a functional group or fusion partner, preferably a heterologous (to the host cell) or synthetic (not native to any cell) functional group or fusion partner. Exemplary groups or partners include, but are not limited to, signal sequences capable of constitutively localizing the expression product to a predetermined subcellular locale such as the Golgi, endoplasmic reticulum, nucleoli, nucleus, nuclear membrane, mitochondria, chloroplast, secretory vesicles, lysosome, and the like; binding sequences capable of binding the expression product to a predetermined protein while retaining bioactivity of the expression product; sequences signaling selective degradation, of itself or co-bound proteins; and secretory and membrane-anchoring signals.

It may also be desirable to provide a partner which conformationally restricts the randomized expression product to more specifically define the number of structural conformations available to the cell. For example, such a partner may be a synthetic presentation structure: an artificial polypeptide capable of intracellularly presenting a randomized peptide as a conformation-restricted domain. Generally such presentation structures comprise a first portion joined to the N-terminal end of the randomized peptide, and a second portion joined to the C-terminal end of the peptide. Preferred presentation structures maximize accessibility to the peptide by presenting it on an exterior loop, for example of coiled-coils, (Myszka and Chaiken (1994) Biochemistry 33:2362). To increase the functional isolation of the randomized expression product, the presentation structures are selected or designed to have minimal biological activity as expressed in the target cell. In addition, the presentation structures may be modified, randomized, and/or matured to alter the presentation orientation of the randomized expression product. For example, determinants at the base of the loop may be modified to slightly modify the internal loop peptide tertiary structure, while maintaining the absolute amino acid identity. Other presentation structures include zinc-finger domains, loops on beta-sheet turns and coiled-coil stem structures in which non-critical residues are randomized; loop structures held together by cysteine bridges, cyclic peptides, etc.

In one embodiment, an assay is a cell-based assay in which a cell that expresses a marker of the invention is contacted with a test compound and the ability of the test compound to up-regulate or down-regulate the marker is determined. Determining the ability of the test compound to up-regulate or down-regulate the ability of the marker to bind to a substrate can be accomplished, for example, by coupling the marker substrate with a radioisotope or enzymatic label such that binding of the marker substrate to the marker can be determined by detecting the labeled marker substrate in a complex. For example, compounds (e.g., marker substrates) can be labeled with ¹²⁵I, ³⁵S, ¹⁴C, or ³H, either directly or indirectly, and the radioisotope detected by direct counting of radio-emission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

It is also within the scope of this invention to determine the ability of a compound (e.g., a marker substrate) to interact with a marker of the invention without the labeling of any of the interactants. For example, a microphysiometer can be used to detect the interaction of a compound with the marker without the labeling of either the compound or the marker (McConnell, H. M. et al. (1992) Science 257:1906). As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and a marker of the invention.

In another embodiment, an assay is a cell-based assay comprising contacting a cell expressing a marker of the invention with a test compound and determining the ability of the test compound to up-regulate or down-regulate expression and/or activity of the marker.

Determining the ability of the test compound to up-regulate or down-regulate an activity of marker can be accomplished, for example, by determining the ability of the marker to bind to or interact with a target molecule of the marker. Determining the ability of a marker of the invention to bind to or interact with a target molecule can be accomplished by one of the methods described herein for determining direct binding. In one embodiment, determining the ability of the marker to bind to or interact with a target molecule can be accomplished by determining the activity of the target molecule. For example, the activity of the target molecule can be determined by detecting induction of a cellular second messenger of the target, detecting catalytic/enzymatic activity of the target and appropriate substrate, detecting the induction of a reporter gene, or detecting a target-regulated cellular response.

In yet another embodiment, an assay of the present invention is a cell-free assay in which a marker of the invention is contacted with a test compound and the ability of the test compound to bind the marker is determined. Binding of the test compound to the marker can be determined either directly or indirectly as described herein. In one embodiment, the assay includes contacting the marker with a known compound which binds the marker to form an assay mixture, contacting the assay mixture with a test compound, and determining the ability of the test compound to interact with the marker, wherein determining the ability of the test compound to interact with the marker comprises determining the ability of the test compound to preferentially bind to the marker as compared to the known compound.

In another embodiment, the assay is a cell-free assay in which a marker of the invention is contacted with a test compound and the ability of the test compound to modulate (e.g., stimulate or inhibit) an activity of the marker is determined. Determining the ability of the test compound to modulate the activity of a marker of the invention can be accomplished, for example, by determining the ability of the marker to bind to a target molecule by one of the methods described herein for determining direct binding. Determining the ability of the marker to bind to a target molecule can also be accomplished using a technology such as real-time Biomolecular Interaction Analysis (BIA) (Sjolander, S. and Urbaniczky, C. (1991) Anal. Chem. 63:2338-2345 and Szabo et al. (1995) Curr. Opin. Struct. Biol. 5:699-705). As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

In an alternative embodiment, determining the ability of the test compound to modulate the activity of a marker of the invention can be accomplished by determining the ability of the marker to further modulate the activity of a downstream effector of a marker target molecule. For example, the activity of the effector molecule on an appropriate target can be determined or the binding of the effector to an appropriate target can be determined as previously described.

In yet another embodiment, a cell-free assay involves contacting a marker of the invention with a known compound which binds the marker to form an assay mixture, contacting the assay mixture with a test compound, and determining the ability of the test compound to interact with the marker, wherein determining the ability of the test compound to interact with the marker comprises determining the ability of the marker to preferentially bind to or modulate the activity of a target molecule.

In more than one embodiment of the above assay methods of the present invention, it may be desirable to immobilize either a marker peptide of the invention or a target molecule of the marker peptide to facilitate separation of complexed from uncomplexed forms of one or both of the proteins, as well as to accommodate automation of the assay. Binding of a test compound to a marker peptide of the invention, or interaction of a marker of the invention with a target molecule in the presence and absence of a candidate compound, can be accomplished in any vessel suitable for containing the reactants. Examples of such vessels include microtiter plates, test tubes, microfuge tubes and the like. In one embodiment, a fusion protein can be provided which adds a domain that allows one or both of the proteins to be bound to a matrix. For example, glutathione-S-transferase/marker peptide fusion proteins or glutathione-S-transferase/target fusion proteins can be adsorbed onto glutathione sepharose beads (Sigma, St. Louis, Mo.) or glutathione-derivatized microtitre plates, which are then combined with the test compound or the test compound and either the non-adsorbed target protein or marker peptide, and the mixture incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described above. Alternatively, the complexes can be dissociated from the matrix, and the level of marker peptide binding or activity determined using standard techniques.

Other techniques for immobilizing proteins on matrices can also be used in the screening assays of the invention. For example, either a marker peptide of the invention or a marker peptide target molecule can be immobilized utilizing conjugation of biotin and avidin or streptavidin. Biotinylated marker peptide or marker peptide target can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce). Alternatively, antibodies reactive with a marker peptide or target that do not interfere with binding of the marker to its target molecule can be derivatized to the wells of the plate, and unbound target or marker peptide trapped in the wells by antibody conjugation. Methods for detecting such complexes, in addition to those described above for the GST-immobilized complexes, include immunodetection of complexes using antibodies reactive with the marker peptide, as well as enzyme-linked assays which rely on detecting an enzymatic activity associated with the marker peptide or marker peptide target molecule.

In another embodiment, modulators of marker expression and/or marker degradation are identified in a method wherein a cell is contacted with a candidate compound and the expression of the marker peptide and/or marker mRNA in the cell is determined. The level of marker peptide and/or marker mRNA in the presence of the candidate compound is compared to the level of marker peptide and/or marker mRNA in the absence of the candidate compound. The candidate compound can then be identified as a modulator of marker peptide expression, marker mRNA expression, and/or marker degradation based on this comparison. For example, when expression of marker peptide and/or marker mRNA is greater and/or the rate of marker degradation is lower (statistically significantly greater or lower, respectively) in the presence of the candidate compound than in its absence, the candidate compound is identified as a stimulator of marker peptide expression and/or marker mRNA expression and/or an inhibitor of marker degradation. Alternatively, when expression of marker peptide and/or marker mRNA is less and/or the rate of marker degradation is higher (statistically significantly lower or greater, respectively) in the presence of the candidate compound than in its absence, the candidate compound is identified as an inhibitor of marker peptide expression and/or marker mRNA expression and/or a stimulator of marker degradation. The level of marker mRNA or peptide expression and marker degradation in the cells can be determined by methods described herein for detecting marker mRNA or peptide.

In yet another aspect of the invention, a marker peptide of the invention can be used as a “bait protein” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO 94/10300), to identify other proteins, which bind to or interact with the marker peptide and are involved in marker peptide activity. Such marker peptide-binding proteins are also likely to be involved in the propagation of signals by marker peptide targets such as, for example, downstream elements of a marker peptide-mediated signaling pathway. Alternatively, such marker peptide-binding proteins are likely to be marker peptide inhibitors.

The two-hybrid system is based on the modular nature of most transcription factors, which consist of separable DNA-binding and activation domains. Briefly, the assay utilizes two different DNA constructs. In one construct, a marker gene of the invention encoding a marker peptide is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a marker peptide-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the marker peptide.

In another embodiment, an assay is an animal model based assay comprising contacting an animal with a test compound and determining the ability of the test compound to up-regulate or down-regulate expression of one or more of the markers of the invention. In one embodiment animals include but are not limited to mammals such as non-human primates, rabbits, rats, mice, and the like and amphibians such as Xenopus.

This invention further pertains to novel agents identified by the above-described screening assays. Accordingly, it is within the scope of this invention to further use an agent identified as described herein in an appropriate animal model as described herein. For example, an agent identified as described herein (e.g., a marker modulating agent) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments of disorders associated with aberrant cellular proliferation as described herein.

Monitoring of Effects During Clinical Trials

Monitoring the influence of agents (e.g., drugs) on the expression or activity of one or more markers of the invention can be applied not only in basic drug screening, but also in clinical trials. For example, the effectiveness of an agent determined by a screening assay as described herein to up-regulate or down-regulate marker gene expression or peptide levels can be monitored in clinical trials of subjects exhibiting aberrant marker gene expression or peptide levels.

In one embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) including the steps of (i) obtaining a pre-administration sample from a subject prior to administration of the agent; (ii) detecting the level of expression of the marker peptide, mRNA or genomic DNA in the pre-administration sample; (iii) obtaining one or more post-administration samples from the subject; (iv) detecting the level of expression or activity of a marker peptide, mRNA or genomic DNA in the post-administration samples; (v) comparing the level of expression or activity of the marker peptide, mRNA, or genomic DNA in the pre-administration sample with the marker peptide, mRNA, or genomic DNA in the post administration sample or samples; and (vi) altering the administration of the agent to the subject accordingly. For example, increased administration of the agent may be desirable to decrease the expression or activity of a marker to lower levels than detected, i.e., to increase the effectiveness of the agent. Alternatively, decreased administration of the agent may be desirable to increase expression or activity of a marker to higher levels than detected, i.e. to decrease the effectiveness of the agent. According to such an embodiment, marker expression or activity may be used as an indicator of the effectiveness of an agent, even in the absence of an observable phenotypic response.

The following examples are provided for exemplification purposes only and are not intended to limit the scope of the invention which has been described in broad terms above.

### EXAMPLE I

### Identification of Genetic Markers Associated With Periodontal Disease

Sub-epithelial connective tissue was harvested from a group of recurrent periodontitis patients and from a group of periodontally well-maintained patients. The samples were stored in RNALATER™ RNA stabilizing reagent (Ambion, Inc., Austin, Tex.) until use. An RNEASY® Mini Kit (QIAGEN® Inc., Valencia, Calif.) was utilized to isolate total RNA from the tissue samples. Isolated RNA was subjected to gene expression profiling using the GENECHIP® Human Genome U133A microarray (Affymetrix, Santa Clara, Calif.) to qualitatively and quantitatively measure gene expression levels. Differential analysis of the microarray data was performed using the “BADGE” program (Bayesian Analysis of Differential Gene Expression).

Out of 22,283 genes investigated, up-regulation of the expression of 68 genes by at least two-fold, and down-regulation of six genes by at least two-fold was observed. The BADGE program gave 93% intrinsic validation and 93% extrinsic validation. These data provide evidence that patients suffering from recurrent periodontitis have multiple up-regulated or down-regulated genes associated with a clinical risk of periodontitis. Accordingly, these 74 genes can be used to diagnose and identify patients with periodontal disease, and to predict future susceptibility to periodontal disease. The 74 genes are presented below.

| Change | Index | Probability | Fold | Lower | Upper |
|---|---|---|---|---|---|
| + | 218154 at | 0.9994130460254655 | 2.647520249300362 | 1.762114925017939 | 4.007967990560 |
| + | 218890 x at | 0.9991644327514897 | 2.455501917019552 | 1.6059001144421492 | 3.773359889359 |
| +LTF | 202018 s at | 0.9990412751141047 | 57.0200120329166 | 8.339838852440105 | 427.7244585392 |
| + | 219257 s at | 0.9987894546089803 | 2.149015484841232 | 1.4501087969433095 | 3.195997916493 |
| + | 212463 at | 0.9984750786195791 | 2.4107161646544126 | 1.520601970672507 | 3.846282736609 |
| + | 221657 s at | 0.9981589458644826 | 2.567421308423699 | 1.682083493037627 | 3.986939788827 |
| +IL-24 | 206569 at | 0.9980879719599725 | 6.531790970309288 | 2.289244108988262 | 19.14705603940 |
| + | 209755 at | 0.9979050838779535 | 2.499439735374874 | 1.5011799319437986 | 4.197494274086 |
| + | 203021 at | 0.9977048829048842 | 2.3058479979714503 | 1.4521607623008765 | 3.695218315612 |
| + | 215890 at | 0.9976331674803336 | 3.9537423284073148 | 1.8347605927949917 | 8.670669905995 |
| + | 221898 at | 0.9976233022246699 | 3.240448090842121 | 1.6560927530694158 | 6.351215745223 |
| + | 209791 at | 0.9974382611927399 | 2.406825821413471 | 1.4715758659451788 | 3.976137499949 |
| + | 220335 x at | 0.997316849755364 | 3.0234406685507635 | 1.7143970519369511 | 5.485388420778 |
| + | 214068 at | 0.9967639110361541 | 2.4076177397566 | 1.4405087683671085 | 4.081630723505 |
| + | 202671 s at | 0.9966656598558259 | 2.1803532054358743 | 1.3692327783821048 | 3.505474111618 |
| +CASP 10 | 210955 at | 0.9965605809444538 | 6.97504293261343 | 2.2594342540890526 | 23.68806349028 |
| + | 201631 s at | 0.9964006133685491 | 2.1732783674834018 | 1.347326980826917 | 3.529339713956 |
| +MMP3 | 205828 at | 0.996188832461379 | 8.256748095267683 | 2.1979763807037527 | 31.99685878520 |
| + | 210022 at | 0.9961441019770203 | 2.6134045226295015 | 1.4263377224324123 | 4.835967841065 |
| + | 217028 at | 0.9959547433626189 | 3.238257245598371 | 1.5153906102228114 | 7.073726624280 |
| + | 208636 at | 0.9956927484246483 | 2.462631390681378 | 1.3765230863940698 | 4.447509046223 |
| + | 203233 at | 0.9956354205954789 | 2.218563227529577 | 1.330098206226082 | 3.735676140040 |
| +FOS B | 202768 at | 0.99525288795394 | 36.90772525029443 | 3.373229024940741 | 412.4843241365 |
| + | 204901 at | 0.9951152885557365 | 2.2423968677567347 | 1.3662216197230448 | 3.747002166006 |
| + | 216018 at | 0.9950078009511835 | 3.5263391852523718 | 1.5814343777175424 | 8.231084778944 |
| + | 205331 s at | 0.9949130504867819 | 2.123317005382675 | 1.2912541217693199 | 3.526368224564 |
| +MMP1 | 204475 at | 0.9947884588231528 | 12.11601318553681 | 2.1814810062835424 | 83.00444524567 |
| + | 219852 s at | 0.9946979550247792 | 4.416646497688851 | 1.6216034232798624 | 12.02921066237 |
| +unknown | 220449 at | 0.9946350084630269 | 4.966046471516079 | 1.6497944878883377 | 15.24908517601 |
| + | 202528 at | 0.9945416338556065 | 2.1743931809816646 | 1.2831187752739686 | 3.721257681638 |
| + | 205513 at | 0.9941928119672386 | 4.271614475969237 | 1.6367022868770857 | 12.70769110783 |
| + | 219866 at | 0.9940260348072285 | 2.7797238442805288 | 1.457224441813055 | 5.431965520490 |
| + | 205680 at | 0.9938935215776177 | 3.2879277802761138 | 1.3747092055858379 | 9.498035628313 |
| + | 205986 at | 0.9938623531176184 | 2.3019672271517893 | 1.2937510659544689 | 4.138342731186 |
| + | 211774 s at | 0.9937328997708801 | 2.5058838084858985 | 1.3363529015369984 | 4.797668361522 |
| + | 214146 s at | 0.993709688242302 | 2.547225885587871 | 1.5110456802162646 | 4.462942048017 |
| + | 220714 at | 0.9936361124304071 | 3.2932834227002434 | 1.4222797019009747 | 7.749006598777 |
| + | 220307 at | 0.9933954257799605 | 2.5169984534117686 | 1.3568685818418325 | 4.797832934970 |
| + | 211964 at | 0.992924428362947 | 2.340688076400559 | 1.294877642833589 | 4.316556837527 |
| + | 210789 x at | 0.9927204622599405 | 2.445208719288133 | 1.2786537129322038 | 4.752743612857 |
| + | 211893 x at | 0.9926992740832654 | 4.647566365739783 | 1.5121933958583869 | 16.24850948843 |
| +ARHGAP8 | 205980 s at | 0.9924787613182522 | 5.102472232621571 | 1.553519621200285 | 16.94437363477 |
| + | 219066 at | 0.9923607936820564 | 2.3878009721166564 | 1.29096326009548 | 4.524039058941 |
| + | 208244 at | 0.9921705485713753 | 2.555131014702481 | 1.2707427932886626 | 5.163474810842 |
| + | 206343 s at | 0.9916533505460814 | 2.4229840109194223 | 1.2902919488230546 | 4.672097068147 |
| + | 203510 at | 0.9915475478626192 | 2.4044825696201095 | 1.3164476212552134 | 4.483013138022 |
| + | 216635 at | 0.9910832702005383 | 2.4818816174136646 | 1.2787997717078827 | 5.004137010659 |
| + | 214192 at | 0.9907699646869721 | 4.536960947998726 | 1.4115272500726046 | 14.73836693451 |
| + | 208496 x at | 0.9907038620011313 | 2.3545762804601975 | 1.2910472328498954 | 4.385920204904 |
| + | 221166 at | 0.990665283134896 | 3.5501489174067076 | 1.3566110122582664 | 9.571691625647 |
| + | 203234 at | 0.9906193392083871 | 3.0379451061202722 | 1.4470246645617177 | 6.701122048993 |
| + | 217560 at | 0.9903185352632184 | 4.1751681138079 | 1.3825748593503726 | 12.92540059698 |
| + | 202388 at | 0.9902322115876568 | 3.0172721815741443 | 1.2944384989299542 | 7.380934398378 |
| + | 209386 at | 0.9899255568802923 | 2.318500967202217 | 1.1991660365929002 | 4.532595124408 |
| + | 213099 at | 0.9899013458647411 | 2.438117212178991 | 1.2456712825161043 | 4.876499883362 |
| + | 208416 s at | 0.9895856429746458 | 2.500279269585169 | 1.2563341125146426 | 5.331442790528 |
| + | 209487 at | 0.9895644806291279 | 2.2816804019537136 | 1.2003439175362813 | 4.433877865716 |
| + | 220522 at | 0.9895152141892336 | 2.25397001918681 | 1.2312026679692023 | 4.239039623148 |
| + | 221009 s at | 0.9894535994432646 | 2.25665686900384 | 1.2188327786829247 | 4.277570064379 |
| + | 209369 at | 0.9893135628407979 | 2.4299137348774815 | 1.20921754044325 | 4.951461886731 |
| + | 202627 s at | 0.9892434868095881 | 3.6676562750289925 | 1.3467867661475195 | 11.00977782246 |
| + | 221237 s at | 0.9888249026478262 | 2.8255123723589284 | 1.225348877736357 | 6.517347780144 |
| +IFI-15K | 205483 s at | 0.9885184868513944 | 5.464522376993452 | 1.3683765661999956 | 21.82222608137 |
| + | 207013 s at | 0.9883325198716619 | 2.6168886742891537 | 1.2277338798942572 | 5.799212197764 |
| + | 213202 at | 0.9880391988377804 | 2.6171828145226153 | 1.2691468224166413 | 5.714458605972 |
| + | 219665 at | 0.9874176602358546 | 2.263113247339441 | 1.392113749397874 | 3.810774171464 |
| + | 215223 s at | 0.9868520871915389 | 2.9262634173371795 | 1.2016978745959752 | 7.496318765494 |
| + | 209959 at | 0.9847598418205218 | 4.621321293474258 | 1.2167938514897516 | 17.55195185765 |
| − | 216229 x at | 0.012566946288261846 | 0.40147137490044493 | 0.19256490468082668 | 0.856373969435 |
| − | 217325 at | 0.010691003371786054 | 0.4118873977171217 | 0.21084555892977527 | 0.833501663986 |
| − | 217974 at | 0.010173090169530313 | 0.17275892947448288 | 0.04321302187188394 | 0.708421566925 |
| − | 207908 at | 0.008529534832871931 | 0.15541644738870491 | 0.04886186219221915 | 0.660734892611 |
| − | 207324 s at | 0.008228355705355182 | 0.08955499126255026 | 0.02014958965953657 | 0.523842977392 |
| − | 221916 at | 0.007087316201577703 | 0.2752558420750297 | 0.11842239861366857 | 0.752347283146 |

In order to validate the microarray data, five up-regulated genes (LTF, MMP-1, MMP-3, IFI-15, MGC5566) and down-regulated genes (KRT2A and DSC-1) were randomly selected for further analysis by real-time PCR. The relative expression level of these genes measured by real-time PCR was similar to those measured by microarrays.

Tables 1 and 2 display the summary of selected genes whose expression was either significantly up-regulated or down-regulated in refractory periodontitis patients. The most commonly used technique in the bioinformatics literature for identifying differential gene expression has been the fold differences, and genes with a fold change greater than a specified threshold, for example >1.7 fold, are generally classified as demonstrating differential expression.

TABLE 1
Up-Regulated Genes

| | | Fold Induction | Fold Induction |
|---|---|---|---|
| | | by Oligonucleotide | by Quantitative |
| Index | Description | Array Hybridization | Real-Time PCR |
| 202018_s_at | LTF | 57 | 39.1 |
| 204475_at | MMP1 | 12 | 22.3 |
| 205828_at | MMP3 | 8.3 | 73.4 |
| 205483_s_at | IFI-15k | 5.5 | 10.2 |
| 220449_at | MGC5566 | 5 | 2 |
| 206569_at | IL-24 | 6.5 | |
| 210955_at | CASP10 | 7 | |
| 202768_at | FOSB | 37 | |
| 205980_s_at | ARHGAP8 | 5.1 | |

TABLE 2
Down-Regulated Genes

| | | Fold Induction | Fold Induction |
|---|---|---|---|
| | | by Oligonucleotide | by Quantitative |
| Index | Description | Array Hybridization | Real-Time PCR |
| 217974_at | TM7SF3 | 0.17 | 3.21 |
| 207908_at | Keratin2a | 0.16 | 0.12 |
| 207324_s_at | Desmocolin1 | 0.09 | 0.045 |

### EXAMPLE II

### Certain Up-Regulated Genetic Markers Identified by Microarray Analysis and BADGE

The following genes were identified as up-regulated in periodontitis samples using the methods set forth in Example I.

Lactotransferrin; LTF

Lactotransferrin has the GenBank Symbol NM_{—}002343. Expression of lactotransferrin was up-regulated in samples obtained from a group of recurrent periodontitis patients approximately 57-fold over lactotransferin expression levels in samples obtained from a group of periodontally well-maintained patients.

V-Fos FBJ Murine Osteosarcoma Viral Oncogene Homolog B; FOSB

V-Fos FBJ murine osteosarcoma viral oncogene homolog B has an Online Mendelian Inheritance in Man identification (OMIM ID) number of 164772. V-Fos FBJ murine osteosarcoma viral oncogene homolog B is also known as: oncogene FOSB; GOSB; and delta-FOSB, included. Expression of V-Fos FBJ murine osteosarcoma viral oncogene homolog B was up-regulated in samples obtained from a group of recurrent periodontitis patients approximately 37-fold over V-Fos FBJ murine osteosarcoma viral oncogene homolog B expression levels in samples obtained from a group of periodontally well-maintained patients.

Matrix Metalloproteinase 1; MMP 1

Matrix metalloproteinase 1 has the OMIM ID Number 120353. Matrix metalloproteinase 1 is also known as: collagenase, fibroblast; CLG; CLGN; and collagenase, interstitial. Expression of matrix metalloproteinase 1 was up-regulated in samples obtained from a group of recurrent periodontitis patients approximately 12-fold over matrix metalloproteinase 1 expression levels in samples obtained from a group of periodontally well-maintained patients.

Matrix Metalloproteinase 3; MMP3

Matrix metalloproteinase 3 has the OMIM ID Number 185250. Matrix metalloproteinase 3 is also known as: stromelysin I; STMY1; STR1; and transin. Expression of matrix metalloproteinase 3 was up-regulated in samples obtained from a group of recurrent periodontitis patients approximately 8.3-fold over matrix metalloproteinase 3 expression levels in samples obtained from a group of periodontally well-maintained patients.

Caspase 10, Apoptosis-Related Cysteine Protease; CASP10

Caspase 10, apoptosis-related cysteine protease has the OMIM ID Number 601762. Caspase 10, apoptosis-related cysteine protease is also known as: MCH4; caspase 10, isoform B, included; CASP10B, included; FADD-like ICE 2, included; FLICE2, included. Expression of caspase 10, apoptosis-related cysteine protease was up-regulated in samples obtained from a group of recurrent periodontitis patients approximately 7-fold over caspase 10, apoptosis-related cysteine protease expression levels in samples obtained from a group of periodontally well-maintained patients.

Interleukin 24; IL24

Interleukin 24 has the OMIM ID Number 604136. Interleukin 24 is also known as: suppression of tumorigenicity 16; ST16; melanoma differentiation-associated gene 7; MDA7. Expression of interleukin 24 was up-regulated in samples obtained from a group of recurrent periodontitis patients approximately 6.5-fold over interleukin 24 expression levels in samples obtained from a group of periodontally well-maintained patients.

Interferon-Induced Protein IFI-15K

Interferon-induced protein IFI-15K has the OMIM ID Number 147571. Interferon-induced protein IFI-15K is also known as: interferon-induced protein 15; IFI15; and G1P2. Expression of interferon-induced protein IFI-15K was up-regulated in samples obtained from a group of recurrent periodontitis patients approximately 5.5-fold over interferon-induced protein IFI-15K expression levels in samples obtained from a group of periodontally well-maintained patients.

ARHGAP8: Rho GTPase Activating Protein 8

ARHGAP8 has the LocusLink ID Number 23779. Expression of ARHGAP8 was up-regulated in samples obtained from a group of recurrent periodontitis patients approximately 5-fold over ARHGAP8 expression levels in samples obtained from a group of periodontally well-maintained patients.

MGC5566: Hypothetical Protein MGC5566

MGC5566 has the LocusLink ID Number 79015. Expression of MGC5566 was up-regulated in samples obtained from a group of recurrent periodontitis patients approximately 5-fold over MGC5566 expression levels in samples obtained from a group of periodontally well-maintained patients.

### EXAMPLE III

### Certain Down-Regulated Genetic Markers Identified By Microarray Analysis and BADGE

The following genes were identified as down-regulated in periodontitis samples using the methods set forth in Example I.

Desmocollin 1; DSC1

Desmocollin 1 has the OMIM ID Number 125643. Expression of desmocollin 1 was down-regulated in samples obtained from a group of recurrent periodontitis patients and expressed at approximately 9% (0.09×) of the level of desmocollin 1 in samples obtained from a group of periodontally well-maintained patients.

Keratin 2A; KRT2A

Keratin 2A has the OMIM ID Number 600194. Keratin 2A is also known as: keratin 2E and KRT2E. Expression of keratin 2A was down-regulated in samples obtained from a group of recurrent periodontitis patients and expressed at approximately 16% (0.16×) of the level of keratin 2A in samples obtained from a group of periodontally well-maintained patients.

Transmembrane 7 Superfamily Member 3; TM7SF3

Transmembrane 7 Superfamily member 3 has the GenBank symbol NM_{—}016551. Expression of transmembrane 7 superfamily member 3 was down-regulated in samples obtained from a group of recurrent periodontitis patients and expressed at approximately 17% (0.17×) of the level of transmembrane 7 superfamily member 3 in samples obtained from a group of periodontally well-maintained patients.

### EXAMPLE IV

### Gene Expression Profiles of Refractory Periodontitis Patients

Patient selection will consist of ten “refractory” periodontitis patients who were treated in a private periodontal practice (New York City) for a minimum of five years following active therapy. An additional ten subjects identified as “periodontally well-maintained” patients with similar base line of disease will be placed in control group. Refractory subjects will be defined as requiring at least three episodes of rescue therapy or three tooth extractions in the five years following active therapy, while periodontally well-maintained patients will be defined as requiring only regular periodontal maintenance care in the five years following active therapy. In addition, refractory subjects will display a continuous recurrence of deep pockets and change in both attachment level and radiographic bone level in spite of receiving conventional periodontal therapy including oral hygiene instruction, scaling and root planing, surgical pocket reduction and regular periodontal maintenance care. All subjects will either never have been smokers or will have stopped smoking at least one year prior to active therapy.

With each patient's consent and following local anesthesia, subepithelial connective tissue (7×7 mm) will be intra-orally harvested aseptically from ten refractory periodontitis patients and ten periodontally well-maintained patients. Tissue samples will be harvested from the active progressing site of disease for the refractory periodontitis patients and from a treated stable site for the well-maintained patients. The RNEASY® Mini Kit (Qiagen® Inc.) will be utilized to isolate total RNA from tissue samples that have been stabilized using RNALATER™ RNA Stabilizing Reagent. Isolated total RNA will be subjected to gene expression profiling using the GeneChip® Human Genome U133A microarray (Affymetrix) to qualitatively and quantitatively measure gene expression levels. Changes of gene expression will be investigated at the level of mRNA using both in silico analysis and laboratory based analysis, e.g., real-time RT-PCR.

### EXAMPLE V

### Gene Expression Profiles of Children and Adolescents Suffering From Aggressive Periodontitis

Children and adolescents can have any of the several forms of periodontitis described in the proceedings of the 1999 International Workshop for a Classification of Periodontal Diseases and Conditions: aggressive periodontitis; chronic periodontitis; and periodontitis as a manifestation of systemic diseases. Although chronic periodontitis is more common in adults, aggressive periodontitis can be more common in children and adolescents. The primary features of aggressive periodontitis include, but are not limited to, a history of rapid attachment and/or bone loss with familial aggregation. Secondary features include, but are not limited to, phagocyte abnormalities and/or a hyper-responsive macrophage phenotype.

Aggressive periodontitis can be localized or generalized. Localized aggressive periodontitis patients typically have interproximal attachment loss on at least two permanent first molars and incisors, with attachment loss on no more than two teeth other than first molars and incisors. Generalized aggressive periodontitis patients typically exhibit generalized interproximal attachment loss including at least three teeth that are not first molars and incisors. In young individuals, the onset of these diseases is often circumpubertal. Localized aggressive periodontitis occurs in children and adolescents without clinical evidence of systemic disease and is characterized by the severe loss of alveolar bone around permanent teeth. Reported estimated of the prevalence of localized aggressive periodontitis in geographically diverse adolescent populations range from 0.1% to 15%.

Longitudinal studies of disease progression in adolescents indicate that subjects with signs of destructive periodontitis at a young age are prone to further deterioration. Such deterioration is often more pronounced at initially affected sites and in individuals diagnosed with juvenile periodontitis and from low socio-economic levels. Deterioration of the periodontal status involves both an increase in extent (i.e., prevalence of lesions within the dentition) and an increase in severity of lesions.

Subepithelial connective tissue will be intra-orally harvested aseptically from ten localized aggressive periodontitis healthy patients, ten refractory periodontitis patients and ten periodontally well-maintained patients. The RNEASY® Mini Kit (Qiagen® Inc.) will be utilized to isolate total RNA from tissue samples that have been stabilized in the RNALATER™ RNA Stabilizing Reagent. Isolated total RNA will be subjected to gene expression profiling using the GeneChip® Human Genome U133A microarray (Affymetrix) to qualitatively and quantitatively measure gene expression levels. Changes in gene expression will be investigated at the level of mRNA using both in silico analysis and laboratory based analysis, e.g., real-time RT-PCR. Such research will further identify the genetic basis of gene expression in periodontal disease; provide simple genetic models for periodontal disease; and provide a set of candidate genes that can serve as novel therapeutic intervention points as well as surrogate and predictive markers of treatment outcome.

### Equivalents

Other embodiments will be evident to those of skill in the art. It should be understood that the foregoing description is provided for clarity only and is merely exemplary. The spirit and scope of the present invention are not limited to the above examples, but are encompassed by the following claims. All publications and patent applications cited above are incorporated by reference herein in their entirety for all purposes to the same extent as if each individual publication or patent application were specifically indicated to be so incorporated by reference.

What is claimed is:

1. A method for diagnosing a periodontal disorder in an organism comprising:
obtaining a biological sample from the organism;
detecting a level of biomarker in the biological sample; and
diagnosing the organism with periodontal disorder based on the level of the biomarker detected, wherein the biomarker is a member selected from the group consisting of lactotransferrin, V-FOS FBJ murine osteosarcoma viral oncogene homolog B, matrix metalloproteinase 1, matrix metalloproteinase 3, caspase 10, interleukin 24, interferon-induced protein IFI-15k, Rho GTPASE activating protein 8, hypothetical protein MGC5566, desmocollin 1, keratin 2A and transmembrane 7 superfamily member 3.

2. A method for diagnosing a periodontal disorder in an organism comprising:
obtaining a biological sample from the organism;
detecting a level of biomarker in the biological sample;
comparing the level of biomarker in the biological sample to a chart correlating a level of biomarker with a periodontal disorder; and
diagnosing the organism with a periodontal disorder when the level of the biomarker in the biological sample corresponds to the level of biomarker that the chart correlates with a periodontal disorder,
wherein the biomarker is a member selected from the group consisting of lactotransferrin, V-FOS FBJ murine osteosarcoma viral oncogene homolog B, matrix metalloproteinase 1, matrix metalloproteinase 3, caspase 10, interleukin 24, interferon-induced protein IFI-15k, Rho GTPASE activating protein 8, hypothetical protein MGC5566, desmocollin 1, keratin 2A and transmembrane 7 superfamily member 3.

3. A method for diagnosing a periodontal disorder in an organism comprising:
obtaining a biological sample from the organism;
detecting a level of biomarker in the biological sample; and
comparing the level of biomarker in the biological sample to a control sample,
wherein the organism is diagnosed with a periodontal disorder when an altered level of the biomarker is detected in the biological sample relative to the control sample,
wherein the biomarker is a member selected from the group consisting of lactotransferrin, V-FOS FBJ murine osteosarcoma viral oncogene homolog B, matrix metalloproteinase 1, matrix metalloproteinase 3, caspase 10, interleukin 24, interferon-induced protein IFI-15k, Rho GTPASE activating protein 8, hypothetical protein MGC5566, desmocollin 1, keratin 2A and transmembrane 7 superfamily member 3.

4. The method of claim 3, wherein the level of biomarker is increased in the biological sample relative to the control sample.

5. The method of claim 3, wherein the level of biomarker is decreased in the biological sample relative to the control sample.

6. A panel of biomarkers for detecting a periodontal disorder in an organism comprising two or more biomarkers selected from the group consisting of lactotransferrin; V-FOS FBJ murine osteosarcoma viral oncogene homolog B, matrix metalloproteinase 1, matrix metalloproteinase 3, caspase 10, interleukin 24, interferon-induced protein IFI-15k, Rho GTPASE activating protein 8, hypothetical protein MGC5566, desmocollin 1, keratin 2A and transmembrane 7 superfamily member 3.

7. A method for monitoring a periodontal disorder in an organism comprising:
obtaining a first biological sample from the organism at a first point in time;
obtaining a second biological sample from the organism at a second point in time;
detecting a level of biomarker in the first and second biological samples; and
comparing the level of biomarker in the first and second biological samples,
wherein a decrease of the level of the biomarker in the second sample relative to the first sample indicates a decrease in a periodontal disorder in the organism, and wherein the biomarker is selected from the group consisting of lactotransferrin; V-FOS FBJ murine osteosarcoma viral oncogene homolog B, matrix metalloproteinase 1, matrix metalloproteinase 3, caspase 10, interleukin 24, interferon-induced protein IFI-15k, Rho GTPASE activating protein 8, and hypothetical protein MGC5566.

8. A method for monitoring a periodontal disorder in an organism comprising:
obtaining a first biological sample from the organism at a first point in time;
obtaining a second biological sample from the organism at a second point in time;
detecting a level of biomarker in the first and second biological samples; and
comparing the level of biomarker in the first and second biological samples,
wherein an increase of the level of the biomarker in the second sample relative to the first sample indicates a decrease in a periodontal disorder in the organism, and wherein the biomarker is selected from the group consisting of desmocollin 1, keratin 2A and transmembrane 7 superfamily member 3.

9. A method for treating a periodontal disorder in an organism comprising:
contacting a cell with an agent that down-regulates a biomarker selected from the group consisting of lactotransferrin, V-FOS FBJ murine osteosarcoma viral oncogene homolog B, matrix metalloproteinase 1, matrix metalloproteinase 3, caspase 10, interleukin 24, interferon-induced protein IFI-15k, Rho GTPASE activating protein 8, and hypothetical protein MGC5566, in a manner to reduce symptoms associated with the periodontal disorder.

10. A method for treating a periodontal disorder in an organism comprising:
contacting a cell with an agent that up-regulates a biomarker selected from the group consisting of desmocollin 1, keratin 2A and transmembrane 7 superfamily member 3, in a manner to reduce symptoms associated with the periodontal disorder.

11. A method for screening compounds useful in treating a periodontal disorder in an organism comprising:
contacting a cell with a test compound and
determining whether the compound up-regulates a biomarker selected from the group consisting of desmocollin 1, keratin 2A and transmembrane 7 superfamily member 3.

12. A method for screening compounds useful in treating a periodontal disorder in an organism comprising:
contacting a cell with a test compound and
determining whether the compound down-regulates a biomarker selected from the group consisting of lactotransferrin, V-FOS FBJ murine osteosarcoma viral oncogene homolog B, matrix metalloproteinase 1, matrix metalloproteinase 3, caspase 10, interleukin 24, interferon-induced protein IFI-15k, Rho GTPASE activating protein 8, and hypothetical protein MGC5566.
